(19) United States
(12) Patent Application Publication
(10) Pub. No.: US 2007/0167706 A1
(43) Pub. Date: Jul. 19, 2007

(54) Method and apparatus for visually supporting an electrophysiological catheter application in the heart by means of bidirectional information transfer

(76) Inventors: Jan Boese, Eckental (DE); Andreas Meyer, Mohrendorf (DE); Marcus Pfister, Bubenreuth (DE); Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/516,138

(22) Filed: Sep. 6, 2006

(30) Foreign Application Priority Data
Sep. 6, 2005 (DE) 10 2005 042 329.9

(51) Int. Cl. A61B 5/05

(52) U.S. Cl. 600/407; 600/424

(57) ABSTRACT

Method and apparatus for visually supporting an electrophysiological catheter application in the heart by means of bidirectional information transfer

The present invention relates to a method and apparatus for visually supporting an electrophysiological catheter application in the heart. For the method, 3D image data of at least the heart, which is captured using a tomographic 3D imaging method prior to execution of the catheter application, and electroanatomical 3D mapping data of at least one area of the heart to be treated, which is captured during execution of the catheter application, is provided and the electroanatomical 3D mapping data and/or at least part of the 3D image data is displayed during execution of the catheter application. The method is characterized in that, in the electroanatomical 3D mapping data and/or the 3D image data, the contour of one or more areas (3) relevant to the catheter application is captured and transferred to the other system in each case on which the areas (3) are superimposed as a single polyline (5) in the representation (2, 4) of the electroanatomical 3D mapping data and/or 3D image data. The method proposed and the associated apparatus provide the user with a rapid overview of the areas relevant to the catheter application.

### CROSS REFERENCE TO RELATED APPLICATIONS

This application claims priority of German application No. 10 2005 042 329.9 filed Sep. 06, 2005, which is incorporated by reference herein in its entirety.

### FIELD OF THE INVENTION

The present invention relates to a method and apparatus for visually supporting an electrophysiological catheter application in the heart, in which 3D image data of at least the heart, which is captured using a tomographic 3D imaging method prior to execution of the catheter application, and electroanatomical 3D mapping data of at least one area of the heart to be treated, which is captured during execution of the catheter application, is provided and the electroanatomical 3D mapping data and/or at least part of the 3D image data is displayed during execution of the catheter application.

### BACKGROUND OF THE INVENTION

The treatment of cardiac dysrhythmias has changed significantly since the introduction of the technique of catheter ablation by means of high-frequency current. In this technique an ablation catheter is introduced under x-ray control into one of the ventricles, via veins or arteries, and obliterates the tissue causing the cardiac dysrhythmias by means of high-frequency current. For catheter ablation to be performed successfully, it is necessary for the cause of the cardiac dysrhythmia to be precisely localized in the ventricle. This localization is effected by means of an electrophysiological investigation in which electrical potentials are recorded in a spatially resolved manner by means of a mapping catheter introduced into the ventricle. This electrophysiological investigation, known as electroanatomical mapping, thus produces 3D mapping data that can be displayed on a monitor. A known electroanatomical 3D mapping method, as may be implemented using the CARTO® system of the company Biosense Webster Inc., USA, is based on electromagnetic principles. Three different weak magnetic alternating fields are built up under the examination table. By means of electromagnetic sensors incorporated in the tip of the mapping catheter it is possible to measure the voltage changes within the magnetic field that are induced by catheter movements, and—with the aid of mathematical algorithms—to calculate the position of the mapping catheter at any point in time. By means of point-by-point mapping of the endocardial contour of a ventricle using the mapping catheter while simultaneously recording the electrical signals, an electroanatomical, three-dimensional map is produced in which the electrical signals are displayed in a color-coded mariner.

The operator orientation required for guiding the catheter has hitherto generally been provided via fluoroscopic visualization. With this technique, as the position of the mapping catheter is known at all times during electroanatomical mapping, after a sufficiently large number of measuring points have been captured orientation can also take place by continuous displaying of the catheter tip in the electroanatomical map, so that fluoroscopic imaging with x-ray radiography can be dispensed with at this stage.

The suboptimal operator orientation possibilities for guiding the catheter constitute a fundamental problem when performing catheter ablation inside the heart.

DE 103 40 544 A1 discloses a method and an apparatus for visually supporting an electrophysiological catheter application in the heart, in which 3D image data of the area of the heart to be treated, which has been captured using a tomographic 3D imaging method prior to execution of the catheter application, is provided and a 3D surface outline of objects in the area to be treated is extracted from the 3D image data by segmentation, the electroanatomical 3D mapping data provided during execution of the catheter application and the 3D image data, preferably one or more ventricles or vessels, which constitutes the 3D surface variation being assigned in a positionally and dimensionally correct manner and displayed superimposed on one another, the 3D image data preferably being displayed via a volume rendering technique or as a polygonal net. Through this superimposing of the 3D surface outline, by means of which the morphology of the area treated can be very well reproduced, on the captured electroanatomical 3D mapping data, the catheter operator is provided with better orientation and more precise details when executing the catheter application than is the case with visual support methods employed hitherto. In addition to the electroanatomical 3D mapping system, the use of such a technique also requires a 3D visualization workstation on which the segmented 3D image data can be displayed in a suitable manner.

### SUMMARY OF THE INVENTION

The object of the present invention is to specify a method and an apparatus for visually supporting an electrophysiological catheter application in the heart, which offers the user a rapid overview of the position and extent of areas relevant to the catheter application.

This object is achieved with the method and apparatus according to the independent claims. Advantageous embodiments of the method and apparatus are the subject matter of the subclaims or may be obtained from the following description and exemplary embodiments.

In the present method, 3D image data, including at least the heart, which is captured using a tomographic 3D imaging method prior to execution of the catheter application, and electroanatomical 3D mapping data of at least one area of the heart to be treated, which is captured during execution of the catheter application, is provided. The electroanatomical 3D mapping data and/or at least part of the 3D image data is visually displayed to the user during execution of the catheter application. The present method is characterized in that, in the 3D image data, a contour of one or more areas is captured, assigned to the electroanatomical 3D mapping data in a positionally and dimensionally correct manner and overlaid into its visual display as a single polyline, and/or that, in the electroanatomical 3D mapping data, a contour of one or more areas is captured, assigned to the 3D image data in a positionally and dimensionally correct manner and overlaid into its visual display as a single polyline.

In the case of the 3D image data, the one or more areas are preferably anatomical structures which, although detectable in the 3D image data, are not detectable in the 3D mapping data. The three-dimensional contour or rather the three-dimensional outline of the relevant area is therefore captured and overlaid into the two-dimensional representation of the 3D mapping data in a positionally and dimensionally correct manner as a single polyline. In the other direction, three-dimensional outlines or contours of areas in the 3D mapping data which are not detectable in the 3D image data are captured. These outlines or contours are also overlaid into the two-dimensional representation of the 3D image data in a positionally and dimensionally correct manner, the present method offering the possibility of displaying either only the electroanatomical 3D mapping data with the one or more overlaid polylines, only the 3D image data with the overlaid polylines or both representations simultaneously or alternately. In the last mentioned case, this corresponds to a bidirectional transfer of corresponding information concerning the position and contour of the relevant areas between the display unit in which the 3D image data is present and the display unit in which the 3D mapping data is present. This generally involves a 3D visualization workstation for the 3D image data and the electroanatomical 3D mapping system for the electroanatomical 3D image data.

Through the superimposition of one or more single polylines which can be both closed polylines enclosing an area and also open polylines e.g. indicating a boundary between two areas, the position and contour of the relevant areas can be visualized in a clear and conspicuous manner for the user in the relevant display. For the catheter application, the user can therefore quickly obtain an overview of areas that are critical or relevant to the application, particularly target areas of the application. This is particularly advantageous in the display of the 3D mapping data, as there the instantaneous position of the catheter is likewise visible because of the continuous updating of this data.

In an advantageous embodiment of the method, the information concerning the position and orientation of the ablation or mapping catheter, which is contained in the data of the electroanatomical 3D mapping system, is likewise transferred in real-time to the display unit for the 3D image data where it is superimposed in a positionally and dimensionally correct manner. This has the advantage that the user can then detect the ablation catheter during the electrophysiological procedure relative to the (compared to the electroanatomical 3D mapping data) very high-resolution preprocedural anatomical 3D image data, it being possible, for example, for the position of the ablation catheter to be displayed as a point and its Orientation as an arrow in the anatomical 3D image data, the display preferably being continuous during the electrophysiological procedure. It is likewise possible to produce an endoscopically rendered (“fly through”) view of the anatomical image data, using the catheter position as the focal point of this view and the catheter orientation as the viewing direction of this view. With regard to these endoscopically rendered views, a focal point can also be selected which is slightly or further behind the current catheter position so that the actual catheter position can be displayed in the endoscopically rendered view. This display of the ablation catheter together with preprocedural 3D image data during the electrophysiological procedure, e.g. on a 3D visualization workstation, is extremely advantageous for the electrophysiologist in respect of an ablation procedure predominantly orientated to anatomical criteria.

The prerequisite for the positionally and dimensionally correct overlaying of the relevant contours is 3D-3D registration of the two 3D coordinate systems of the electroanatomical 3D mapping data and the anatomical 3D image data. For 3D-3D registration of this kind, different methods are known, such as those described in the already mentioned DE 103 40 544 A1, whose disclosure content in relation thereto is included in the present patent application. 3D-3D registration in which the surface of the ventricle to be treated is extracted from the electroanatomical 3D mapping data and from the anatomical 3D image data and matched has been found to be particularly advantageous here. A landmark-based coarse pre-registration can be used as a starting value for this surface matching. This registration technique, also described in the above-mentioned publication, allows 3D-3D registration during the electrophysiological procedure in real-time. If the relevant registration information is then present only on the electroanatomical 3D mapping system or only on the display unit for the 3D image data, this data can of course be transferred to the other system in each case. This also applies in the case of registration updates which may be necessary particularly if the patient moves during the electrophysiological procedure.

In many 3D mapping systems, the electroanatomical 3D mapping data is present in coordinates which are not determined relative to the origin of the coordinate system of the 3D mapping system but relative to a reference position sensor which can be attached e.g. to the patient's back. This means that the coordinates of the surface points of the 3D mapping data are insensitive to patient movements. This can be utilized for the present method by using the coordinate system of the reference sensor as coordinate system of the 3D mapping data for 3D-3D registration, thereby enabling the position and contour of the relevant areas to be transferred in these reference coordinates to the 3D image data, as the latter is registered with respect to the reference coordinate system.

X-ray computer tomography, magnetic resonance tomography or 3D ultrasound imaging methods can be used for acquiring the 3D image data. Combinations of said imaging methods are also possible. It should merely be ensured that the 3D images are recorded in the same cardiac phase and/or respiratory phase as the electroanatomical 3D mapping data provided, in order to capture the same cardiac state in each case. This can be ensured using the known techniques of ECG gating or respiration gating for acquiring the image data and the electroanatomical mapping data.

The present apparatus comprises an electroanatomical 3D mapping system for acquiring and displaying electroanatomical 3D mapping data, which system is connected via a data link to a 3D visualization workstation for displaying 3D image data. The electroanatomical 3D mapping system and/or the 3D visualization workstation incorporate a determination module which is designed to determine the contour of one or more areas in the electroanatomical 3D mapping data or the 3D image data. In both systems there is additionally provided a transfer module which is designed to transmit the contour and position data in one or both directions between the two systems. The electroanatomical 3D mapping system and/or the 3D visualization workstation additionally incorporate a registration module which is designed for 3D-3D registration of the electroanatomical 3D mapping data or the electroanatomical 3D mapping system and the 3D image data. The electroanatomical 3D mapping system and/or the 3D visualization workstation also comprise a visualization module which displays the electroanatomical 3D mapping data or the 3D image data on a screen and overlays data received from the other system concerning the position and contour of one or more areas into the relevant display in a positionally and dimensionally correct manner as one or more polylines of the basis of the registration information.

The relevant positions and contours can be transferred continuously and automatically throughout the electrophysiological procedure. If required, the transfer can alternatively be initiated by user interaction. The overlaying of the one or more polylines itself can take place in different ways, e.g. in color, dashed, flashing or as a filled contour.

### BRIEF DESCRIPTION OF THE DRAWINGS

The present invention and the associated apparatus will now be explained again in greater detail with reference to exemplary embodiments in conjunction with the associated drawings without limiting the protective scope defined by the claims:

FIG. 1 shows an example of the transfer and overlaying of a contour into a display of the 3D image data,

FIG. 2 shows an example of the transfer and overlaying of a contour into a display of the 3D mapping data,

FIG. 3 shows an example of the transfer and overlaying of the position and orientation of the ablation catheter into an endoscopically rendered display of the 3D image data, and

FIG. 4 schematically illustrates the apparatus for carrying out the method.

### DETAILED DESCRIPTION OF THE INVENTION

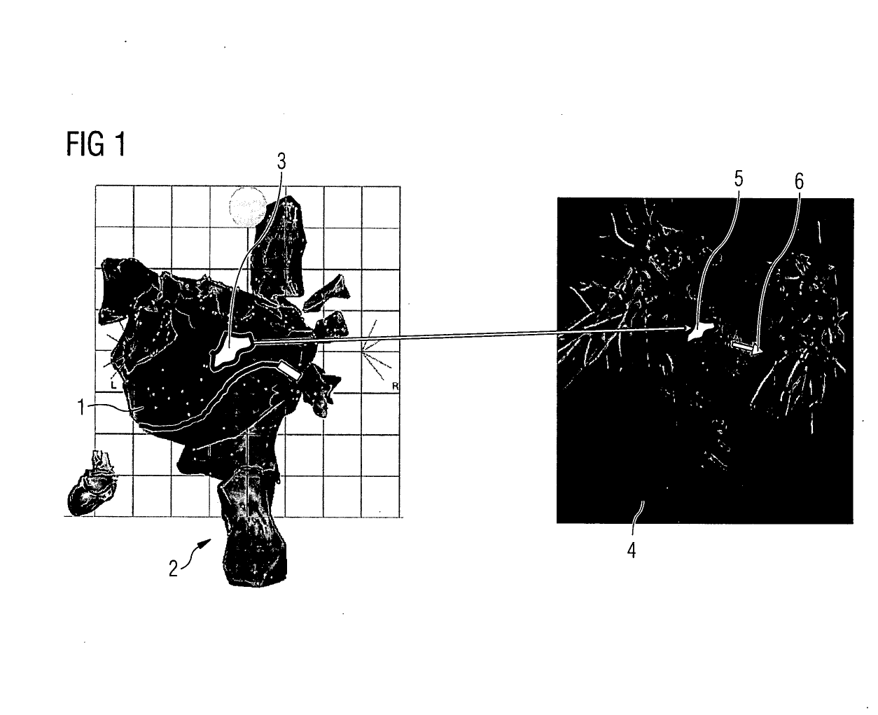

FIG. 1 shows an example of transferring the contour of an area 3 captured in the electroanatomical mapping data from the electroanatomical mapping system to the 3D visualization workstation on which a representation 4 of the 3D image data is displayed. During the electrophysiological procedure, an electroanatomical 3D map 2, as shown in the left-hand part of FIG. 1, is visualized on the electroanatomical 3D mapping system. This representation is based on a simplified model of the heart on which the individual 3D mapping data are visible as mapping points 1. In this representation the position and orientation of the mapping catheter 9, which can be derived from the 3D mapping data, is additionally superimposed. In this 3D mapping data, the position and contour of a three-dimensional area 3 is now captured which is visible in the left-hand part of the figure, this possibly being, for example, the outline of post-infarction scarring of the left ventricle which can be determined from the electroanatomical 3D mapping data. This determination can take place both interactively and automatically. The position and contour are transferred from the electroanatomical 3D mapping system to the 3D visualization workstation on which the 3D image data is visualized in this example in a volume rendering representation. In this example, this representation 4 of the 3D image data shows the surface of the heart as it appears in the right-hand section of FIG. 1.

The data transferred to the 3D visualization workstation from the electroanatomical 3D mapping system can then be overlaid into the displayed 3D anatomy on the basis of previously performed 3D-3D registration. It is overlaid as a single polyline 5, as can be seen in the figure. The area enclosed by this polyline can additionally be hatched or monochromatically highlighted. In the example in FIG. 1, the position and orientation of the mapping catheter 9 is simultaneously transferred to the 3D visualization workstation where it is superimposed using the arrow 6.

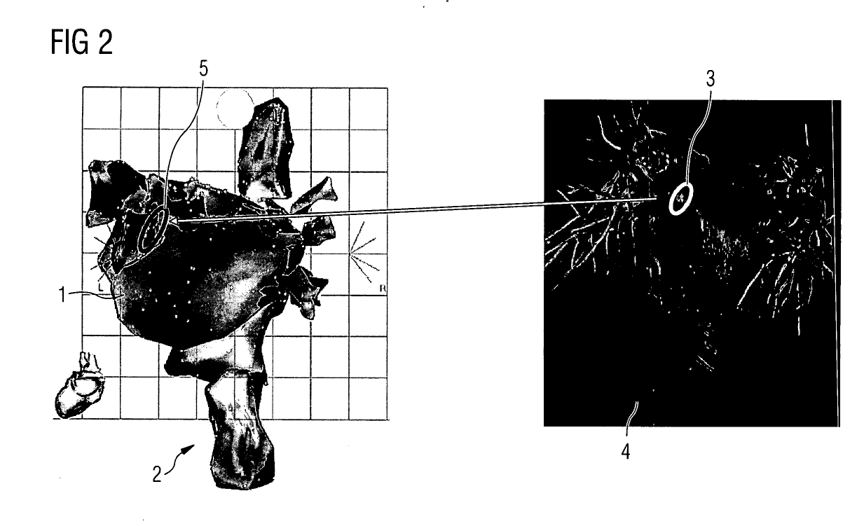

FIG. 2 shows an example in which information is transferred in the opposite direction. In this example, anatomical structures which are important for the electrophysiological catheter application are identified in the 3D image data in order to capture their contour and position. This can be seen in the right-hand section of FIG. 2 which shows a representation 4 of the 3D image data of the heart as a surface view. In the image data, the position and contour of the corresponding area 3 is captured and transferred to the electroanatomical 3D mapping system where the contour of this area is overlaid as a polyline 5 in a positionally and dimensionally correct manner, on the basis of the 3D-3D registration, into the 3D map 2, i.e. into the representation of the electroanatomical 3D mapping data.

In this way the contour of the esophagus, for example, which has been extracted from the anatomical 3D image data at the 3D visualization workstation can be transferred to the electroanatomical 3D mapping system and then overlaid into the electroanatomical 3D mapping using the 3D-3D registration information. The superimposition of the esophagus is useful, as there is a risk of esophageal perforation during ablation procedures on the posterior wall of the left atrium.

A further example of an area relevant to the catheter application or orientation during the catheter application are pulmonary vein ostia which can be visualized with high resolution in the preprocedural 3D image data. These are identified prior to the electrophysiological procedure, e.g. interactively as a procedural planning step, and their position and contour are transferred to the electroanatomical 3D mapping system where the corresponding polyline 5 is then superimposed during the electrophysiological procedure so that the ablation catheter can be guided along the overlays which identify the pulmonary vein ostia. This corresponds to the example in FIG. 2.

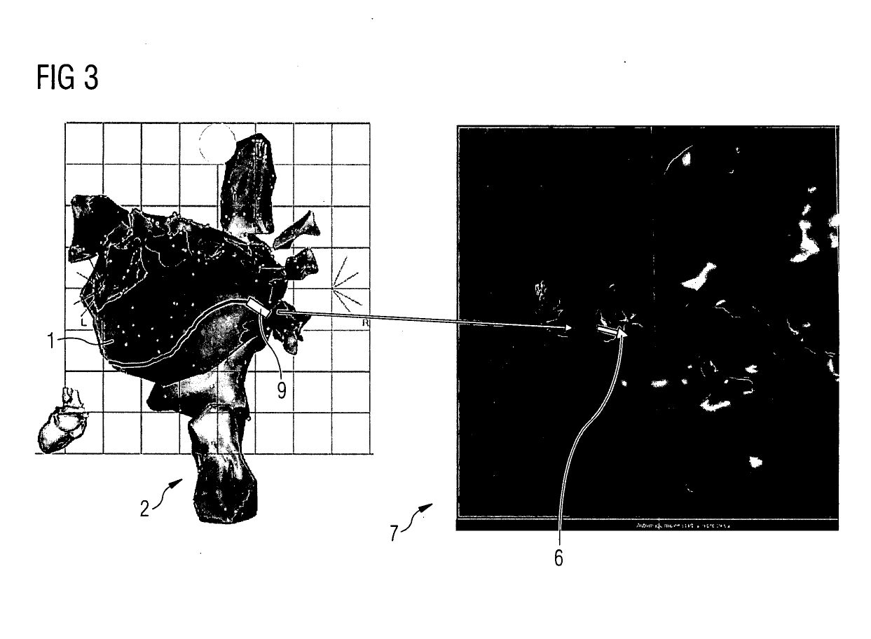

The possibility of additionally overlaying the instantaneous position and orientation of the ablation catheter into the representation 4 of the 3D image data has already been explained in connection with FIG. 1. FIG. 3 shows by way of example another possibility for indicating the position of the ablation catheter in the display of the 3D image data on the 3D visualization workstation, an endoscopically rendered (“fly through”) view 7 of the anatomical 3D image data being generated in which a focal point is selected which is behind the current catheter position. The current catheter position and orientation is likewise shown by an arrow 6 in this view.

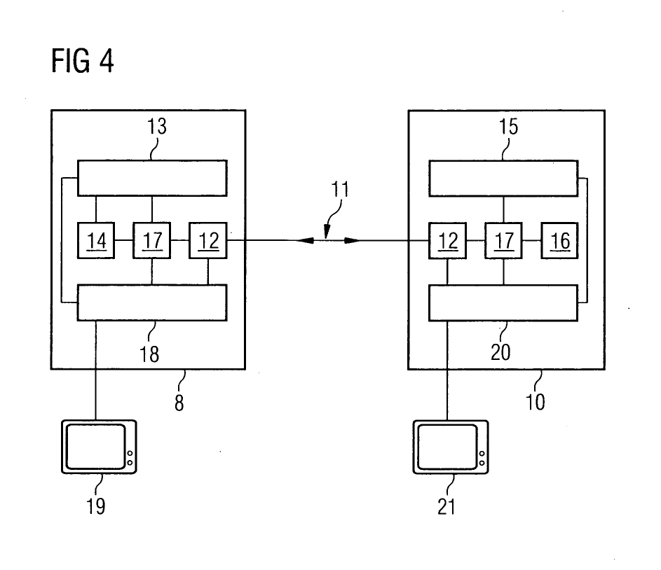

Lastly, FIG. 4 schematically illustrates an example of the structure of the present apparatus which is comprised of an electroanatomical 3D mapping system 8 and a 3D visualization workstation 10. The two systems are interconnected via a data link 11 via which the corresponding image data, position and contour data as well as registration information can be transferred. For this purpose both systems incorporate a transfer module 12 via which data transfer takes place. In this example, bidirectional transfer of data between the two systems 8, 10 is assumed, the electroanatomical 3D mapping system 8 incorporating a determination module 14 which extracts corresponding contours from the electroanatomical 3D mapping data which are stored in a memory 13 and transmits them to the transfer module 12 for transfer to the 3D visualization workstation 10. In the same way, the 3D visualization workstation 10 comprises a determination module 16 which extracts corresponding contours from the 3D image data stored in a memory 15 and transfers them via the transfer module 12 to the electroanatomical 3D mapping system 8. Both systems additionally incorporate a registration module 17 which is designed to register the image or mapping data of the two systems or to store the corresponding registration information. In the visualization module 18 of the electroanatomical mapping system 8, the data transferred by the 3D visualization workstation 10 is lastly processed to overlay the corresponding areas as a single polyline into a representation of the 3D mapping data on a monitor 19. In the same way, corresponding overlaying of one or more polylines by the visualization module 20 of the 3D visualization workstation 10 takes place on a monitor 21.

With an apparatus of this kind, contours of three-dimensional areas can therefore be transferred bidirectionally between an electroanatomical 3D mapping system and a 3D visualization workstation after 3D-3D registration of an electroanatomical 3D map with preprocedurally recorded anatomical 3D image data, the relevant contours being overlaid as single polylines into the corresponding representations of the electroanatomical 3D mapping data or 3D image data. This overlaying can also take place during the procedure in real-time together with the superimposition of the position and orientation of the catheter.

For positionally and dimensionally correct assignment, a gating technique which takes the corresponding physiological parameters (ECG, respiration) into account should be used for recording the relevant data, gating information being understood as information concerning the phase of the one or more physiological parameters. For example, in the case of ECG gating, the gating information can be transferred as a percentage relative to the complete cardiac cycle or as an absolute time value relative to the start of a cardiac cycle. Depending on the available gating and the transfer direction, a distinction can be drawn between the following eight cases:
Electroanatomical 3D map without gating/3D image data without gating/transfer direction to the 3D visualization workstation: the generation of electroanatomical 3D maps without gating only appears to be useful if the complete 3D map is generated once as a complete image and not by subsequent sampling of individual surface points. In this case of no physiological gating, no further action is necessary in respect of contour transfer/superimposition. The information that the electroanatomical 3D map has been recorded without gating is transferred to the 3D visualization workstation.
Electroanatomical 3D map without gating/3D image data without gating/transfer direction to the electroanatomical 3D mapping system: the information that the 3D image data has been recorded without gating is transferred to the electroanatomical 3D mapping system.
Electroanatomical 3D map with gating/3D image data without gating/transfer direction to the 3D visualization workstation: the gating information of the 3D map is transferred to the 3D visualization workstation where it can be displayed.
Electroanatomical 3D map with gating/3D image data without gating/transfer direction to the electroanatomical 3D mapping system: the information that the 3D image data has been recorded without gating is transferred to the electroanatomical 3D mapping system.
Electroanatomical 3D map without gating/3D image data with gating/transfer direction to the 3D visualization workstation: the generation of electroanatomical 3D maps without gating only appears useful if the entire 3D map is generated once as a complete image and not by subsequent sampling of individual surface points. In this case of generating a one-time complete image, no gating of the electroanatomical 3D map is required. However, the physiological gating factor can be determined in parallel with acquiring the 3D image map and transferred to the 3D visualization workstation as “gating info”. The gating information received at the 3D visualization workstation is handled at the 3D visualization workstation as described in the next but one variant.
Electroanatomical 3D map without gating/3D image data with gating/transfer direction to the electroanatomical 3D mapping system: the gating information is transferred from the 3D visualization workstation to the 3D mapping system where it can be displayed.
Electroanatomical 3D map with gating/3D image data with gating/transfer direction to the 3D visualization workstation: the contours to be transferred relate to a particular cardiac phase at which the electroanatomical 3D map was generated. This cardiac phase is transferred as gating information with the contours to the 3D visualization workstation. If the anatomical image data is present at the 3D visualization workstation as 4D image data (or 5D, 6D, etc. in the case of a plurality of the physiological gating parameters), the corresponding 3D image dataset which best fits the electroanatomical 3D map because of its cardiac phase can be determined from this 4D image data using the gating information. Contour overlaying is then performed there. The overlaying of the contour can then also be performed in a time varying (4D) visualization corresponding to the gating information: thus the contour can be displayed in a different way (in respect of brightness, color, flashing frequency, etc.) if the transferred gating information best coincides with the gating information of the currently displayed 3D image (from the 4D series). It is also possible to continuously vary the representation of the contour: the overlaying is varied using the time offset between the two items of gating information, e.g. brighter and brighter the better the items of gating information correspond to one another.
Electroanatomical 3D map with gating/3D image data with gating/transfer direction to the electroanatomical 3D mapping system: the contours to be transferred relate to a particular heart phase at which the anatomical 3D image data was generated. The gating information is transferred to the electroanatomical 3D mapping system where it can be used to adjust the gating during mapping so that it matches the gating of the anatomical image data as closely as possible. Analogously to the preceding variant, the contour overlay (flashing frequency, brightness, color, etc.) can be varied in the 4D maps according to the transferred gating information.

1-18. (canceled)

19. A method for visually supporting an electrophysiological catheter application in a heart of a patient during a medical procedure, comprising:
recording a 3D image data of the heart using a tomographic 3D imaging method;
determining a contour of an area relative to the catheter application in the 3D image data;
executing the catheter application;
capturing and displaying an electroanatomical 3D mapping data of a region of the heart to be treated during executing the catheter application;
assigning the contour to the electroanatomical 3D mapping data positionally and dimensionally correctly;
overlaying the contour into a visual representation of the electroanatomical 3D mapping data as a single polyline; and
displaying the visual representation of the electroanatomical 3D mapping data with the overlaid polyline.

20. The method as claimed in claim 19, wherein the contour is:
an anatomical structure of a pulmonary vein ostia or an esophagus of the patient, or
an outline of a post-infarction scarring of the patient.

21. The method as claimed in claim 19, wherein a contour of another area relative to the catheter application is determined in the electroanatomical 3D mapping data and assigned to the 3D image data positionally and dimensionally correctly and overlaid into a visual representation of the 3D image data as another single polyline.

22. The method as claimed in claim 21, wherein the contour is an outline of a post-infarction scarring of the patient.

23. The method as claimed in claim 21, wherein the visual representation of the electroanatomical 3D mapping data and the visual representation of the 3D image data are displayed exclusively, simultaneously, or alternately.

24. The method as claimed in claim 19, wherein the positionally and dimensionally correct assignment is performed automatically by using:
an artificial marker which is attached to a chest of the patient prior to recording the 3D image data and is visible both in the 3D image and mapping data, or
a distinctive anatomical point which is visible both in the 3D image and mapping data, or
a surface matching via extracting a 3D surface outline from the 3D image data and coinciding approximately with a 3D surface outline from the 3D mapping data.

25. The method as claimed in claim 24, wherein the positionally and dimensionally correct assignment is performed automatically in a first stage during executing the catheter application based on the artificial marker or the distinctive anatomical point and refined in a second stage by the surface matching.

26. The method as claimed in claim 19, wherein a position and orientation of a catheter used for the catheter application is determined from the 3D mapping data and displayed in the visual representation of the 3D image data.

27. The method as claimed in claim 19, wherein the 3D mapping and image data are each captured using a physiological gating technique which the polyline is displayed differently depending on a gating instant of the 3D mapping and a gating instant of the 3D image data if the two gating instants are identical or different.

28. The method as claimed in claim 27, wherein the polyline varies with a time offset between the two gating instants.

29. The method as claimed in claim 19, wherein at least part of the 3D image data is displayed during executing the catheter application.

30. A method for visually supporting an electrophysiological catheter application in a heart of a patient in a medical procedure, comprising:
recording a 3D image data of the heart using a tomographic 3D imaging method;
executing the catheter application;
capturing and displaying an electroanatomical 3D mapping data of at least a region of the heart to be treated during executing the catheter application;
determining a contour of an area from the electroanatomical 3D mapping data;
assigning the contour to the 3D image data positionally and dimensionally correctly; and
overlaying the contour into a visual representation of the 3D image data as a single polyline; and
displaying the visual representation of the 3D image data with the overlaid polyline.

31. The method as claimed in claim 30, wherein part of the 3D image data is displayed during executing the catheter application.

32. An apparatus for visually supporting an electrophysiological catheter application in a heart of a patient in a medical procedure, comprising:
an electroanatomical 3D mapping system that captures and displays an electroanatomical 3D mapping data of at least a region of the heart of the patient to be treated;
a 3D visualization workstation that displays a 3D image data of the heart of the patient; and
a data link that connects the electroanatomical 3D mapping system and the 3D visualization workstation,
wherein the 3D visualization workstation comprises a determination module that determines a contour of an area relative to the catheter application in the 3D image data and a transfer module that transmits the contour of the area to the electroanatomical 3D mapping system via the data link,
wherein the electroanatomical 3D mapping system comprises a visualization module that overlays the transferred contour as a single polyline into a representation of the 3D mapping data positionally and dimensionally correctly based on a 3D-3D registration.

33. The apparatus as claimed in claim 32, wherein the electroanatomical 3D mapping system comprises another determination module that determines another contour of another area relative to the catheter application in the electroanatomical 3D mapping data and the transfer module that transmits the another contour to the 3D visualization workstation via the data link,
wherein the 3D visualization workstation comprises another visualization module that overlays the transferred contour as another single polyline into a representation of the 3D image data positionally and dimensionally correctly based on the 3D-3D registration.

34. The apparatus as claimed in claim 32, wherein the 3D-3D registration is performed automatically by a positionally and dimensionally correct assignment based on:
an artificial marker or a distinctive anatomical point which is visible both in the 3D image and mapping data, or
a surface matching of a 3D surface outline from the 3D image data with a 3D surface outline from the 3D mapping data.

35. The apparatus as claimed in claim 34, wherein the 3D-3D registration is performed automatically in a first stage based on the artificial marker or anatomical point and is refined in a second stage by the surface matching.
